(19) United States
(12) Patent Application Publication
(10) Pub. No.: US 2023/0358747 A1
(43) Pub. Date: Nov. 9, 2023

(54) IN VITRO METHOD FOR THE DETECTION OF SARS-COV-2 IN A SAMPLE FROM THE UPPER RESPIRATORY TRACT USING A COLORIMETRIC IMMUNOSENSOR AND RELATED COLORIMETRIC IMMUNOSENSOR

(71) Applicant: TECHNOGENETICS S.P.A., Milano (IT)

(72) Inventors: Stefania AMOROSI, Piacenza (IT); Sergio BOLLETTI CENSI, Napoli (IT); Antonio CERVO, Tavazzano con Villavesco (Lodi) (IT); Bartolomeo DELLA VENTURA, Maddaloni (Caserta) (IT); Elena SAUCHELLI, Venticano (Avellino) (IT); Salvatore SESSO, Montella (Avellino) (IT); Raffaele VELOTTA, Napoli (IT)

(21) Appl. No.: 18/246,148

(22) PCT Filed: Sep. 21, 2021
(86) PCT No.: PCT/IB2021/058575

§ 371 Date: Mar. 21, 2023

(30) Foreign Application Priority Data
Sep. 22, 2020 (IT) 102020000022333

(51) Int. Cl. G01N 33/569; G01N 33/543; G01N 21/78

(52) U.S. Cl. CPC G01N 33/56983; G01N 33/54346; G01N 21/78; G01N 2333/165

(57) ABSTRACT

An in vitro method and a kit for detecting the SARS-CoV-2 virion using a colorimetric immunosensor in a biological sample from the upper respiratory tract of a subject is provided. The biological sample is a nasopharyngeal swab sample. The in vitro method and kit are based on the use of gold capture nanoparticles carrying on their surface at least one antibody capable of binding a SARS-CoV-2 surface antigen.

### FIELD OF THE INVENTION

The present invention relates to an in vitro method for the detection of SARS-CoV-2 in a biological sample from the upper respiratory tract by using a colorimetric immunosensor and the related colorimetric immunosensor.

### STATE OF THE ART

In the field of Research and Diagnostics, colorimetry-based optical biosensors are becoming increasingly important due to their versatility, ease of use, and ability to reach an extremely low limit of detection (LOD) of the analyte.

Known among the optical biosensors are colorimetric biosensors based on gold nanoparticles, which use a physical property of gold nanoparticles, designated as Localized Surface Plasmon Resonance (LSPR), to monitor the colour change when different-size clusters are formed following the interaction between the analyte to be detected and the gold nanoparticles.

Optical transducers are particularly interesting for the direct detection (label free) of microorganisms. These sensors are designed to detect minimal conversions in refractive index or thickness that occur when cells attach to receptors immobilized on the surface of the transducer, correlating changes in concentration, mass, or number of molecules to direct changes in light characteristics. The sensing mechanism is based on the conversion of signals resulting from binding to the target to be detected into physical signals which can be amplified and detected. Nanomaterials, which are characterized by extremely small dimensions and to which suitable surface modifications can be made, allow highly specific interaction with biomolecular targets, thus showing enormous potential in the field of biological detection.

More generally, gold nanoparticles find application in a wide range of disciplines, including magnetic fluids, catalysis, biotechnology/biomedicine, magnetic resonance imaging, and environmental remediation. In most of these applications, better functionality of these nanoparticles is seen when their size is less than a critical value, which depends on the material but typically is approximately 10-20 nm.

Biosensors based on specificity of interaction between an antigen and the corresponding antibody for determining the analyte of interest are commonly referred to as immunosensors. The antibody immobilization procedure is a crucial step in the construction of these devices since the orientation of the antibody molecules on the electrode surface significantly affects the performance of a biosensor. In fact, the formation of a layer of antibodies with their binding sites well oriented and facing the antigen improves the efficiency of the biosensor, making the choice of the immobilization method one of the most important aspects to be taken into account in the construction of an immunosensor. Generally, antibody immobilization methods involve physical or chemical adsorption of these molecules.

The method using ionic or electrostatic bonds, hydrophobic interactions and van der Waals bonds between the antibody and the surface, and not requiring chemical modifications to the protein (Sharma, Byrne, and Kennedy 2016; Um et al. 2011) is mentioned among the simplest adsorption procedures. The main disadvantage of the above method is that the antibodies are randomly oriented and may therefore not properly expose the antigen binding sites.

More effective methods of immobilization of antibodies are based on the formation of covalent bonds between the antibody and the gold surface (Alves, Kiziltepe, and Bilgicer 2012; Ho et al. 2010; Vashist et al. 2011; Rahman et al. 2007). For example, biotinylated antibodies can be immobilized on surfaces modified with streptavidin or avidin (Barton et al. 2009; Ouerghi et al. 2002) or the antibodies can be immobilized on surfaces modified with proteins such as protein A or protein G (J. E. Lee et al. 2013; Inkpen et al. 2019; Sharafeldin and Rusling 2019; Fowler, Stuart, and Wong 2007). Eventually, methods of immobilization of antibodies involving the trapping in polymer matrices have been developed in the last decade (Sun et al. 2011; Bereli et al. 2013; Moschallski et al. 2013; Yamazoe 2019).

Among the possible immobilization strategies, the formation of self-assembled monolayers (SAMs) is one of the most widely used methods for the construction of immunosensors. For example, the oriented immobilization of an antibody on the gold surface of an electrode can be achieved by exploiting the formation of SAMs of thiol carboxylic acids (Barreiros dos Santos et al. 2013; Malvano, Pilloton, and Albanese 2018; Wan et al. 2016) or by immobilizing the antibodies on electrochemically deposited cysteamine layers (Malvano, Pilloton, and Albanese 2018). In addition, the use of cross-linking agents such as glutaraldehyde, specifically for the immobilization of anti-E. coli antibodies on a polyaniline substrate, has also recently been reported with interesting results in the detection of this bacterium (Chowdhury et al. 2012). Therefore, SAMs are widely used as linkers for the immobilization of antibodies in an oriented manner on a gold surface, but despite the numerous advantages they exhibit in various applications, there are still several aspects that should be taken into consideration in order to understand and control their physical and chemical properties (Vericat et al. 2010; Mandler and Kraus-Ophir 2011; Chaki and Vijayamohanan 2002). A self-assembled monolayer on gold surfaces is commonly represented as a perfect monolayer in which the molecules are in a perfectly packed configuration. Actually, this idea is far from reality, and quality control of a SAM is a crucial point in many applications. The construction of a well assembled monolayer strongly depends on the purity of the chemical reagents and solutions used, and even the presence of a minimum amount of contaminants, such as for example thiolated molecules which are typical impurities in thiol compounds, can lead to a non-uniform and therefore non-ideal layer (C. Y. Lee et al. 2005).

In recent years, different types of immunosensors have been described in studies published in the literature.

Iarossi, M. et al (2018) (“Colorimetric Immunosensor by Aggregation of Photochemically Functionalized Gold Nanoparticles” ACS Omega 3, 4, 3805-3812) describes a colorimetric immunosensor that uses the phenomenon of surface plasmon resonance of gold nanoparticles, as well as the application of this system for the detection of human IgG immunoglobulins.

Liu Y., et al (2015) (“Calorimetric detection of influenza A virus using antibody-functionalized gold nanoparticles” Analyst 140(12)3989-3995) investigated the use of a colorimetric immunosensor based on gold nanoparticles modified with anti-haemagluttinin monoclonal antibodies to determine the influenza A virus. However, no evidence of clinical effectiveness of the immunosensor described is provided in this document.

The research described in Della Ventura B. et al (2020) (“Colorimetric Test for Fast Detection of SARS-CoV-2 in Nasal and Throat Swabs” MedRixv doi: https://doi.org/10.1101/2020.08.15.20175489 and ACS Sensors 5, 3043-3048) concerns the use of a colorimetric immunosensor for the detection of SARS-CoV-2 coronavirus in a nasopharyngeal swab.

The ongoing serious pandemic caused by the novel Severe Acute Respiratory Syndrome Coronavirus 2 (SARS-CoV-2) has posed major public health challenges in many countries. Due to the lack of specificity of symptoms of the serious disease caused by the new coronavirus, called COVID-19, confirmation of diagnosis requires laboratory tests to be performed on respiratory and/or serum samples from patients. Large-scale diagnostic tests also play a key role in isolating asymptomatic COVID-19 patients in an attempt to stem the spread of infection.

Among the procedures currently used for the diagnosis of SARS-CoV-2 coronavirus infection, the method based on reverse transcription polymerase chain reaction (RT-PCR) plays a primary role. This method allows the identification of the viral genome in samples from the upper respiratory tract, in particular in samples taken using nasopharyngeal swabs. However, the diagnostic application of the PCR has major limitations due to the complexity of execution and timing, as well as the need for dedicated instrumentation and trained personnel.

The immunological approaches that have been developed for the diagnosis of COVID-19 also exhibit significant problems. For example, lateral flow assays, while allowing for rapid analysis of many samples, are characterized by low sensitivity.

Therefore, there is a need to provide diagnostic tests which allow for rapid identification of the SARS-CoV-2 virus through simple procedures, while maintaining high specificity and sensitivity parameters.

These and other objects are achieved by the in vitro method and the related kit as defined in the attached independent claims, which are suitable for detecting the SARS-CoV-2 virion in a biological sample from the upper respiratory tract of a subject.

Additional features of the invention are identified in the dependent claims, which form an integral part of the specification.

As will be clear from the following detailed description, the in vitro method according to the invention allows the diagnostic result to be obtained in a very short time, within the minute range, and requires minimum quantities of the sample to be analysed, within a range of approximately one millilitre volume, which are very simple to collect. The particular simplicity of the procedure, which does not require any sophisticated instrumentation, also allows costs to be significantly reduced.

Therefore, a first object of the present invention is an in vitro method for the detection of the SARS-CoV-2 virion in a biological sample from the upper respiratory tract of a subject, comprising the steps of:
(a) resuspending the biological sample in a buffer solution, thereby obtaining a sample solution;
(b) taking a portion from said sample solution and contacting said portion with a colloidal suspension of gold capture nanoparticles carrying on their surface at least one antibody capable of binding a SARS-CoV-2 surface antigen, the antigen being selected from the group consisting of the membrane protein (M), envelope protein (E), spike protein (S), and any combination thereof, thereby obtaining a reaction mixture;
(c) determining the formation of a cluster of gold nanoparticles on the surface of the SARS-CoV-2 virion in the reaction mixture, said cluster resulting from the interaction between said antibody and said antigen, the determination being carried out by detecting a change in an optical parameter of the reaction mixture, said change in an optical parameter of the reaction mixture being indicative of the presence of the SARS-CoV-2 virion in the biological sample from the upper respiratory tract.

Within the scope of the present description, the term “virion” refers to the mature viral particle, including the genome, nucleocapsid, and envelope thereof.

The method according to the invention is based on the Localized Surface Plasmon Resonance (LSPR) physical principle consisting in the occurrence of coherent and non-propagating oscillations of free electrons in metal particles following irradiation with an electromagnetic wave, the frequency of which resonates with the surface plasmon. The resonance of the surface plasmon, which gives the colloidal solution its colour, depends on various factors, such as the size of the nanoparticles, and can change considerably when the nanoparticles are in contact with each other, or in any case at a distance much smaller than their own diameter. Generally, the coupling of metal nanoparticles, for example gold nanoparticles, to one another in a colloidal suspension occurs through formation of dimers, trimers, or larger chains, up to the formation of clusters, and involves a change in the plasmon resonance, and therefore in the colour of the solution, which can even be detected with the naked eye.

According to the invention, the clustering of the gold nanoparticles occurring in the reaction mixture is mediated by a biological mechanism consisting in the specific interaction between the antibodies immobilized on the surface of said capture nanoparticles and the corresponding antigens occurring on the SARS-CoV-2 virus surface. Therefore, clustering only occurs if the SARS-CoV-2 viral particle is present in the test sample, thus providing extreme specificity to the method of the invention.

Biological samples from the upper respiratory tract suitable for use in the method according to the invention are preferably selected from the group consisting of nasal swab sample, nasopharyngeal swab sample, pharyngeal swab sample or oropharyngeal swab sample. A nasopharyngeal swab sample is particularly preferred.

The procedure for collecting said samples involves taking mucus and secretions lining the surface of the mucous membranes of the nasopharynx or oropharynx by using a sterile swab, which may be, for example, a small stick carrying a tip made of cotton or a synthetic material such as, for example, rayon.

The method of the invention provides that the biological sample of the upper respiratory tract, after sampling, is resuspended in a buffer solution, preferably in a saline buffer solution optionally containing one or more antimicrobial agents such as, for example, the Copan Universal Transport Medium (UTM).

The selection of a suitable buffer solution for resuspending the biological sample from the upper respiratory tract falls within the skills of those of ordinary skill in the art.

As indicated above, the metal nanoparticle used in the method according to the invention is a gold nanoparticle.

Preferably, the gold nanoparticle has a diameter ranging from 1 nm to 100 nm, more preferably from 2 nm to 40 nm. Most preferred is a gold nanoparticle having a diameter of nm.

In the method according to the invention, the at least one antibody on the surface of the capture gold nanoparticle is capable of binding a SARS-CoV-2 surface antigen selected from the group consisting of the membrane protein (M), envelope protein (E), spike protein (S), and any combination thereof.

As is known in the art, the above-mentioned viral proteins contribute together to the formation of the external viral envelope. Among these, the spike protein is responsible for the binding of SARS-CoV-2 to the host cell by favouring the fusion of the viral envelope with the cell membrane.

Among the antibody molecules suitable for use in the method according to the invention, monoclonal or polyclonal antibodies, monomeric (Fab) or dimeric (F(ab′)2) antibody fragments, single chain antibody fragments (scFv), or any binding protein derived from an antibody scaffold are mentioned by way of non-limiting example.

According to the invention, it is contemplated that anti-SARS-CoV-2 antibodies as defined above may be present on the capture gold nanoparticle, in any possible combination. The proximity of said antibodies to the surface of the gold nanoparticle allows the distance between the nanoparticles of the cluster to be minimal, thus allowing the occurrence of the LSPR phenomenon.

Methods suitable for achieving the immobilization of one or more antibody molecules in the immediate vicinity on the surface of a gold nanoparticle are known in the art, even if they are essentially limited to physisorption or to a photochemical technique (Photochemical Immobilization Technique, PIT) in which the immobilization of the antibodies on the gold surface in the correct orientation is achieved by irradiating these molecules with UV light [Della Ventura B. et al. (2019) “Biosensor surface functionalization by a simple photochemical immobilization of antibodies: experimental characterization by mass spectrometry and surface enhanced Raman spectroscopy”. Analyst 144, 6871-6880].

Unlike physisorption, the PIT method allows a robust and durable functionalization, allowing industrial application of the method.

The selection of the most appropriate antibody immobilization method for use within the scope of the present invention falls well within the skills of those of ordinary skill in the art.

Preferably, the amount of gold capture nanoparticles as defined above in the colloidal suspension is in the range of 1 to 10²⁰ nanoparticles (np)/ml based on the total volume of the suspension, more preferably 10 to 10¹⁵ np/ml. In an even more preferred embodiment, the amount of gold capture nanoparticles is 10¹⁰ np/ml based on the total volume of the suspension.

Advantageously, the method according to the invention allows the SARS-CoV-2 viral particle to be detected in its entirety in the test sample due to the ability of the gold nanoparticles carrying antibodies specifically directed against the viral surface proteins to cluster on the surface of the virion by keeping very close to each other and forming a layer around it. Therefore, thanks to the specific determination of active viral particles, the method according to the invention is particularly suitable for identifying cases in which a SARS-CoV-2 infection is still ongoing.

As illustrated above, in the method according to the invention the clustering of gold nanoparticles on the surface of the SARS-CoV-2 virion is determined by detecting a change in an optical parameter of the reaction mixture.

In one embodiment of the method of the invention, the detected change in the optical parameter is a colour change of the reaction mixture that is detectable by the naked eye.

In this embodiment, a further optional step consists in comparing the detected colour of the reaction mixture with a colorimetric scale. This step increases the interpretative quality of the result.

Also importantly and advantageously, the embodiment illustrated above does not require the use of instrumentation.

In another embodiment of the invention, the detected change in the optical parameter is a reduction in the transmittance value of the reaction mixture measured at a predetermined wavelength in the visible range, preferably at 560 nm.

Within the scope of the present description, the expression “wavelength in the visible range” refers to a wavelength between approximately 390 nm and approximately 760 nm.

According to the above embodiment, the measurement of the transmittance value of the reaction mixture can be carried out by using a photometer or colorimeter instrument, preferably calibrated with a standard solution having a transmittance value of 100%.

In yet another embodiment of the invention, the detected change in the optical parameter is an increase in the absorbance value of the reaction mixture measured at a predetermined wavelength in the visible range, preferably at 560 nm. Suitable instrumentation for carrying out the above absorbance measurement is a spectrophotometer, for example, which can be a portable or benchtop spectrophotometer.

According to a further embodiment, the detected change in the optical parameter is an increase in the area under the absorption spectrum of the reaction mixture in a wavelength range between 200 nm and 700 nm.

In this embodiment, the method according to the invention allows a quantitative measurement which is indicative of the SARS-CoV-2 viral load in the test sample. According to this embodiment, the use of a standard curve in addition makes it possible to obtain an “absolute” measure of the viral load.

As previously stated, a kit including means suitable to perform the method according to the invention is also included within the scope of the present invention.

Therefore, a second aspect of the present invention is a diagnostic kit for the detection of the SARS-CoV-2 virion in a biological sample from the upper respiratory tract of a subject, the kit comprising:
(i) a buffer solution suitable for resuspending the biological sample; and
(ii) a colloidal suspension of gold capture nanoparticles carrying on their surface at least one antibody capable of binding a SARS-CoV-2 surface antigen, the antigen being selected from the group consisting of the membrane protein (M), envelope protein (E), spike protein (S), and any combination thereof.

In one embodiment, the diagnostic kit of the invention also comprises a support containing a colorimetric scale, for example a colorimetric strip.

In another embodiment, the diagnostic kit of the invention also comprises a portable colorimeter or photometer.

Preferably, the portable photometer is equipped with a tungsten lamp and a monochromator capable of isolating the wavelength at 560 nm.

Preferably, the portable colorimeter is equipped with a diode capable of emission at 560 nm.

Among the portable instruments suitable for use in the kit of the invention, the portable model HI96759 photometer and the portable model HI759 colorimeter, both from Hanna Instruments, are mentioned as examples.

In the diagnostic kit of the invention, the colloidal suspension comprising the gold capture nanoparticles can be dispensed into a plurality of single disposable test tubes.

Alternatively, said colloidal suspension can be supplied in a single package, for example in a dedicated dropper device.

The following experimental examples are provided for illustrative purposes only. Therein, reference is made to the accompanying drawings, wherein:

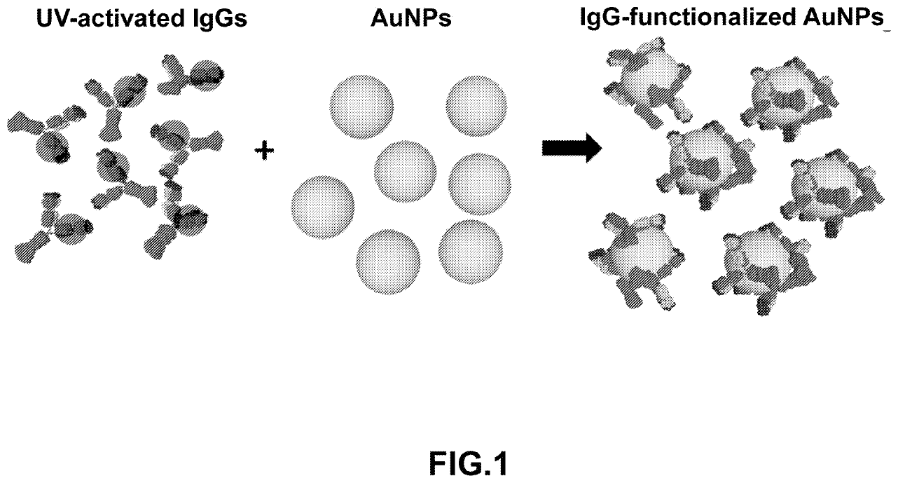

FIG. 1 is a schematic representation of the method for functionalizing the surface of gold nanoparticles with type G immunoglobulins (Photochemical Immobilization Technique, PIT). IgG antibodies are irradiated with UV rays using a lamp of appropriate power, causing the reduction of disulfide bridges at specific positions in the light chain constant part of the antibody. The production of thiols allows the formation of a covalent bond between the antibody and the surface of the gold nanoparticle, leaving one of the two antigen-recognition portions of the antibody free.

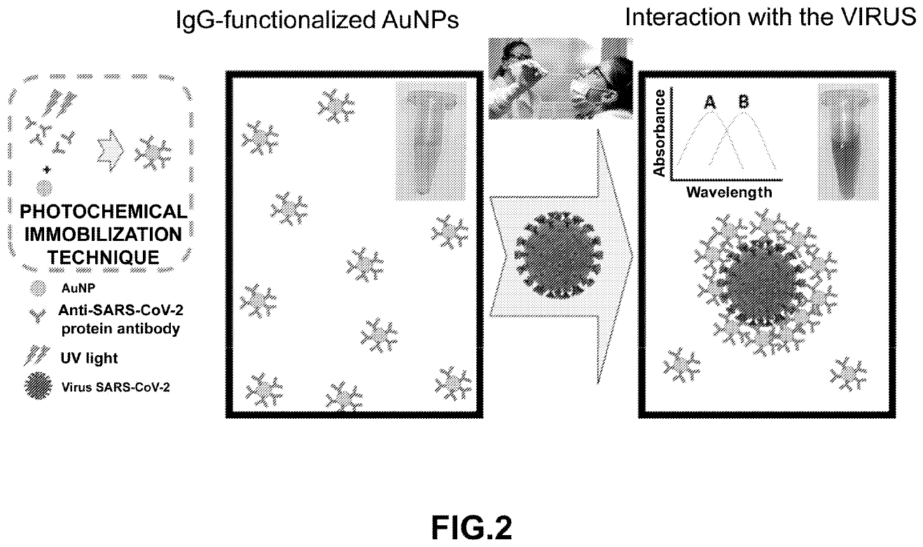

FIG. 2 is a schematic representation of the method of the invention. The colloidal suspension of gold nanoparticles functionalized with anti-SARS-CoV-2 antibodies is contacted with the sample containing the virus, thus forming a reaction mixture. Following the clustering of the functionalized gold nanoparticles around the viral particle, the reaction mixture changes colour. As the concentration of viral particles increases, the shift towards blue increases.

### EXPERIMENTAL SECTION

### Example 1: Preparation of the Colloidal Gold Solution (Synthesis of Nanoparticles)

For their experiments, the present inventors obtained the synthesis of gold nanoparticles having a diameter of approximately 20 nm using a variant of a protocol known in the art (the Turkevich method). According to this protocol, tetrachloroauric acid is first solubilized in water and the addition of sodium citrate causes reduction of the gold, resulting in the production of a gold seed and subsequently the growth of gold around it. The synthesis reaction consisted in mixing 1 mL of HAuCl4 (10 mg/mL) and 2 mL of sodium citrate dihydrate (25 mg/mL) in 100 mL of milliQ (ultrapure) water. The operating temperature was maintained at 90° C., with gentle stirring. The formation of gold nanoparticles was identified by a drastic change in the colour of the solution from yellow to orange.

At the end of the synthesis, the solution was centrifuged at 6 G for 30 minutes, thereby obtaining gold nanoparticles ready to be functionalized.

### Example 2: Functionalization

The surface of the gold nanoparticles was functionalized by using the mechanism known as Photochemical Immobilization Technique (PIT), as described in FIG. 1.

Briefly, IgG antibodies directed against the membrane protein (Membrane, M), the envelope protein (Envelope, E), and the spike protein (S) of the SARS-CoV-2 virus were used (0.1 mg/mL).

A quartz cuvette containing the antibody solution at a concentration of 1 μg/mL was inserted into a specially designed UV lamp and irradiated with UV rays for 30 seconds in order to obtain the reduction of some disulfide bridges in specific positions of the antibody. Subsequently, gold nanoparticles having a diameter of 20 nm in size were functionalized, obtaining a concentration of nanoparticles with the anti-envelope antibody of 10¹⁰ nanoparticles (np)/mL, a concentration of nanoparticles with the anti-spike antibody of 10¹⁰ np/mL, and a concentration of nanoparticles with the anti-membrane antibody of 10¹⁰ np/mL. Any empty spaces left on the gold nanoparticles were then blocked by using a solution containing BSA (50 μg/mL).

Finally, the colloidal suspensions containing the three different antibodies were mixed together in a ratio of 1:1:1 so as to obtain a single suspension of gold nanoparticles carrying the three anti-SARS-CoV-2 antibodies, thus significantly increasing the specificity of the system.

Purification of the obtained samples was carried out by centrifugation at 6 G for 10 minutes.

### Example 3: Sample Preparation

The nasopharyngeal swab, after collection using an appropriate stick, was resuspended in Universal Transport Medium (UTM) (Copan Diagnostics, Inc.), an isotonic solution commonly used to transport samples in which the presence of viruses is to be detected. Among various ingredients, the UTM buffer contains: Hank's balanced salt solution, bovine serum albumin, L-cysteine, gelatin, sucrose, L-glutamic acid, HEPES buffer, phenol red, vancomycin, amphotericin B, and colistin.

### Example 4: Detection

Gold capture nanoparticles prepared and functionalized as described above were used for the SARS-CoV-2 virus detection experiments.

Briefly, after performing the nasopharyngeal swab, a volume of 100 μL of the buffer solution containing the virus was added and mixed with the colloidal suspension of capture gold nanoparticles for about 1 minute. A colour change occurred in the reaction mixture that went from orange to blue passing through purple. In particular, the shift towards blue increased with increasing concentration of virus. The simple buffer solution in which no colour change was seen was used as a negative control.

For transmittance or absorbance analysis, a defined volume of the nasopharyngeal swab, after being resuspended, was deposited by a sterile disposable Pasteur pipette in a test tube (Wheaton type) already containing the gold capture nanoparticle suspension.

Alternatively, defined volumes of the test sample and the gold capture nanoparticle suspension were dispensed together into a supplied cuvette.

The reaction mixture obtained with the preparation procedures described above was mixed by stirring the test tube/cuvette, or by pipetting, thereby allowing the formation of the clusters of functionalized gold nanoparticles on the surface of the SARS-CoV-2 virion.

Subsequently, the test tube or cuvette was housed in the supplied portable reader, which provided absorbance or transmittance values indicative of the positivity/negativity of the sample, i.e., the presence or absence of SARS-CoV-2 viral particles.

In the experiments carried out by the present inventors, a portable photometer was used, which was equipped with a tungsten lamp and a monochromator capable of isolating the wavelength at 560 nm, after suitable calibration with a standard. A simple “blank”, i.e., a sample assigned a 100% transmittance or 0% absorbance, was used as the standard. Disposable cuvettes containing the reaction mixture were used for the photometric analysis.

In their experiments, the present inventors alternatively used a colorimeter device equipped with a diode capable of emitting at 560 nm and, similarly to the above-mentioned device, of returning a transmittance value. In this procedural mode, after being resuspended, the nasopharyngeal swab sample was dispensed and assayed directly in the disposable reaction tube pre-packaged with the colloidal suspension of gold capture nanoparticles.

The transmittance values measured in the experiments described above are indicative of the amount of SARS-CoV-2 viral particles present in the test sample.

In order to measure the absorbance of the reaction mixture, the inventors also used a benchtop spectrophotometer instrument which is capable of emitting in a wide spectrum of wavelengths ranging, for example, from 200 to 700 nm. The absorbance measurements taken at the different wavelengths allowed the area under the absorption spectrum to be calculated, thus providing a “relative” quantitative determination of the viral load. The viral load measurement can become “absolute” by means of a calibration of the described technique.

What is claimed is:

1. An in vitro method for detecting the SARS-CoV-2 virion in a biological sample from the upper respiratory tract of a subject, the method comprising:
a) resuspending the biological sample in a buffer solution, thereby obtaining a sample solution;
b) taking a portion from said sample solution and contacting said portion with a colloidal suspension of gold capture nanoparticles carrying on their surface at least one antibody capable of binding a SARS-CoV-2 surface antigen, the SARS-CoV-2 surface antigen being selected from the group consisting of membrane protein (M), envelope protein (E), spike protein (S), and any combination thereof, thereby obtaining a reaction mixture; and
c) determining the formation of a cluster of the gold capture nanoparticles on the surface of the SARS-CoV-2 virion in the reaction mixture, said cluster resulting from an interaction between said at least one antibody and said SARS-CoV-2 surface antigen, the determination being carried out by detecting a change in an optical parameter of the reaction mixture, said change in the optical parameter of the reaction mixture being indicative of the presence of the SARS-CoV-2 virion in the biological sample from the upper respiratory tract.

2. The in vitro method of claim 1, wherein the change in the optical parameter is a colour change of the reaction mixture detectable by the naked eye.

3. The in vitro method of claim 2, further comprising comparing the detected colour of the reaction mixture with a colorimetric scale.

4. The in vitro method of claim 1, wherein the change in the optical parameter is a reduction in the transmittance value of the reaction mixture measured at a predetermined wavelength in the visible range.

5. The in vitro method of claim 1, wherein the change in the optical parameter is an increase in the absorbance value of the reaction mixture measured at a predetermined wavelength in the visible range.

6. The in vitro method of claim 1, wherein the change in the optical parameter is an increase in the area under the absorption spectrum of the reaction mixture in a wavelength range between 200 nm and 700 nm.

7. The in vitro method of claim 1, wherein the biological sample from the upper respiratory tract is selected from the group consisting of nasal swab sample, nasopharyngeal swab sample, pharyngeal swab sample and oropharyngeal swab sample.

8. The in vitro method of claim 1, wherein the change in the optical parameter is detected by a colorimeter or a photometer.

9. A diagnostic kit for detecting the SARS-CoV-2 virion in a biological sample from the upper respiratory tract of a subject, the kit comprising:
(i) a buffer solution suitable for resuspending the biological sample; and
(ii) a colloidal suspension of gold capture nanoparticles carrying on their surface at least one antibody capable of binding a SARS-CoV-2 surface antigen, the SARS-CoV-2 surface antigen being selected from the group consisting of membrane protein (M), envelope protein (E), spike protein (S), and any combination thereof.

10. The diagnostic kit of claim 9, wherein the colloidal suspension is dispensed into a plurality of single disposable test tubes.

11. The diagnostic kit of claim 9, further comprising a support containing a colorimetric scale.

12. The diagnostic kit according to of claim 9 or 10, further comprising a portable colorimeter or a photometer.

13. The in vitro method of claim 4, wherein the change in the optical parameter is a reduction in the transmittance value of the reaction mixture measured at 560 nm.

14. The in vitro method of claim 5, wherein the change in the optical parameter is an increase in the absorbance value of the reaction mixture measured at 560 nm.
